(19) United States
(12) Patent Application Publication
(10) Pub. No.: US 2016/0228385 A1
(43) Pub. Date: Aug. 11, 2016

(54) PURIFIED CBD AND CBDA, AND METHODS, COMPOSITIONS AND PRODUCTS EMPLOYING CBD OR CBDA

(71) Applicants: Robert E. Sievers, Boulder, CO (US); Lia Rebits, Thornton, CO (US)

(72) Inventors: Robert E. Sievers, Boulder, CO (US); Lia Rebits, Thornton, CO (US)

(73) Assignee: COLORADO CAN LLC, Boulder, CO (US)

(21) Appl. No.: 15/017,384

(22) Filed: Feb. 5, 2016

Related U.S. Application Data
(60) Provisional application No. 62/158,908, filed on May 8, 2015.
(60) Provisional application No. 62/112,695, filed on Feb. 6, 2015.
(60) Provisional application No. 62/112,616, filed on Feb. 5, 2015.

(51) Int. Cl. A61K 31/05; C07F 5/00; A61K 9/20; A61K 36/185; A61K 9/14

(52) U.S. Cl. CPC A61K 31/05; A61K 36/185; A61K 9/146; A61K 9/2027; C07F 5/003

(57) ABSTRACT

A purified cannabidiol (CBD) extract and/or cannabidiolic acid (CBDA) extract is isolated from industrial hemp and comprises less than 0.5 wt % organic impurities as measured by HPLC and ¹H NMR spectroscopy exhibits no detectable peak at 4.07 ppm as measured by ¹H NMR spectroscopy. The CBD and/or CBDA extract is in crystalline form. The CBD extract exhibits a melting point as measured by differential scanning calorimetry (DSC) of 69-70° C. Dry powder compositions comprise such extracts. Additional dry powder compositions comprise polyvinylpyrrolidone and an amorphous CBD extract. An adduct comprises CBD and/or CBDA bonded to a paramagnetic trivalent lanthanide (III) metal chelate.

### FIELD OF THE INVENTION

The present invention is directed to purified cannabidiol (CBD) extracts and purified cannabidiolic acid (CBDA) extracts, and to methods for producing such extracts, and compositions and products comprising CBD and/or CBDA, including, but not limited to dry powder compositions, single unit oral dosages, and adducts of CBD or CBDA with paramagnetic trivalent lanthanide (III) metal chelates.

In the present disclosure, the term “Cannabis plant” encompasses wild type Cannabis sativa and also variants thereof, including cannabis chemovars or cultivars which naturally contain different amounts of the individual cannabinoids, Cannabis sativa subspecies indica, including the variants var. indica and var. kafiristanica, Cannabis indica, and also plants which are the result of genetic crosses, self-crosses or hybrids thereof. The term “Cannabis plant material” is to be interpreted accordingly as encompassing plant material derived from one or more cannabis plants and includes dried cannabis biomass.

### BACKGROUND OF THE INVENTION

Cannabidiol (CBD) is the decarboxylated product of cannabidiolic acid (CBDA) and results from heating CBDA at about 130° C. CBD is of the formula:

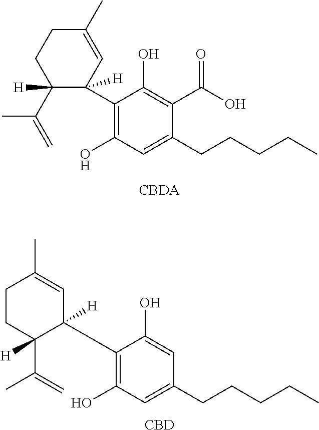

CBD has been demonstrated to be a promising and effective treatment for substance use disorders. This has been demonstrated in both pre-clinical studies as well as clinical trials on human subjects. These studies have shown CBD to reduce drug-seeking behavior and withdrawal symptoms resulting from chronic use and addiction to commonly abused substances including opiates (cocaine, heroin, morphine), nicotine and marijuana (Crippa et al, J Clin Pharm Ther, 38(2):162-4 (April 2013)). Preliminary clinical trials using CBD to treat nicotine addiction have been highly promising. In human subjects seeking to quit smoking, CBD administered via an inhaler reduced cigarette use by 40% compared to no reduction for subjects administered a placebo inhaler (Morgan et al, Addict Behav, 38(9):2433-6 (September 2013)). These studies demonstrate the effective use of CBD to treat substance abuse disorders and addiction to commonly abused substances.

Additional studies of medical uses of CBD have also been directed to treatment of epileptic seizures, uncontrollable seizures in pediatric patients, cancer treatment, reducing adverse effects of other cancer treatments, pain management, and treatment of auto-immune disorders, among others. See, for example, Friedman et al, N Engl J Med, 373:1048-1058 (Sep. 10, 2015). Nora Volkow, the Director of the National Institute of Drug Abuse (NIDA), testified in the US Senate Caucus on International Narcotics Control on Jun. 24, 2014 that CBD (and other cannabinoids) have a range of effects that may be therapeutically useful including anti-oxidant, anti-seizure, neuroprotective, anti-inflammatory, analgesic, anti-tumor, and anti-anxiety properties. Treatment of multiple sclerosis, Parkinson's disease, alcohol abuse, tumor metastasis, post-traumatic stress disorders were also documented in humans or animal models.

However, medical use of CBD or CBDA, as well as other cannabinoids, is complicated by a lack of standardization in both composition and methods of delivery, as well as poorly known degradation pathways of the various cannabinoids. Accordingly, improved methods of obtaining purified CBD and CBDA, and compositions and products comprising CBD and/or CBDA for research and medicinal purposes are desired.

### SUMMARY OF THE INVENTION

Accordingly, it is an object of the invention to provide purified CBD extracts and/or CBDA extracts, and to provide compositions and products comprising CBD extract and/or CBDA extract, and methods of producing purified products and compositions.

In one embodiment, the invention is directed to a cannabidiol (CBD) extract or a cannabidiolic acid (CBDA) extract isolated from industrial hemp, and comprising less than 0.5 wt % organic impurities as measured (1) by high performance liquid chromatography (HPLC) at 30° C., and (2) by proton nuclear magnetic resonance CH NMR) spectroscopy at 300 megahertz using a 0.1 wt % solution of the CBD or CBDA extract in deuterated chloroform solution relative to a tetramethylsilane internal standard. The CBD or CBDA is in crystalline form and a 0.1 wt % solution of the extract in deuterated chloroform exhibits no detectable peak at 4.07 ppm, relative to a tetramethylsilane internal standard, as measured by ¹H NMR spectroscopy at 300 megahertz.

In another embodiment, the invention is directed to a dry powder composition of such an extract.

In another embodiment, the invention is directed to a dry powder composition comprising polyvinylpyrrolidone (PVP) and a CBD extract or a CBDA extract isolated from industrial hemp, wherein the CBD or CBDA is amorphous.

In another embodiment, the invention is directed to a method of producing a dry powder composition, the method comprising mixing at least one carrier, an extract containing CBD or CBDA and a supercritical or near supercritical fluid, and rapidly reducing the pressure on the mixture, whereby droplets are formed, and passing the droplets through a flow of heated gas.

In another embodiment, the invention is directed to a method of purifying a CBD extract or a CBDA extract in oil form, the method comprising dissolving the oil extract in near-supercritical carbon dioxide and removing a precipitated impurity exhibiting a peak at 4.07 ppm relative to a tetramethylsilane internal standard, as measured by proton nuclear magnetic resonance (¹H NMR) spectroscopy at 300 megahertz.

In another embodiment, the invention is directed to method of sterilizing a CBD extract or a CBDA extract, the method comprising dissolving the extract in liquid carbon dioxide, pressurizing the solution to a pressure in a range of 2000 to 3000 psi, and repeatedly increasing and decreasing the pressure of the solution in the range of 2000 to 3000 psi.

In another embodiment, the invention is directed to an adduct comprising CBD or CBDA bonded to a paramagnetic trivalent lanthanide (III) metal chelate.

These and additional aspects of the invention and the advantages thereof will be more fully described in and apparent from the detailed description.

### BRIEF DESCRIPTION OF THE DRAWINGS

The present disclosure will be more fully understood in view of the Drawings, in which:

FIG. 1 shows the HPLC chromatogram of a Cannabis extract as described in Example 1.

FIGS. 2A-2F show the ultraviolet (UV) spectra of the minor peak materials in FIG. 1 as described in Example 1.

FIG. 3 shows the HPLC chromatogram of a Cannabis extract as described in Example 2.

FIGS. 4A-4D show the HPLC chromatograms of a Cannabis extract processed in the manners as described in Example 3.

FIGS. 5A-5E show the results of analytical analysis of CBD extracts as described in Example 4.

FIGS. 6A and 6B show the results of ¹H NMR and X-ray diffraction analyses, respectively, of a CBD-containing dry powder composition as described in Example 7.

FIGS. 7A-7D show the results of analytical analysis of an Eu(fod)₃-CBD adduct, as well as comparative materials, as described in Example 8.

Additional details of the drawings and specific embodiments of the invention will be more fully apparent in view of the Detailed Description and the Examples.

### DETAILED DESCRIPTION

The invention is directed to purified Cannabis materials, and more specifically, to purified cannabidiol (CBD) extracts and purified cannabidiolic acid (CBDA) extracts, methods for purifying such extracts, and compositions and products comprising purified CBD or CBDA.

In specific embodiments, the invention is directed to a cannabidiol (CBD) extract or a cannabidiolic acid (CBDA) extract isolated from industrial hemp, and comprising less than 0.5 wt % organic impurities as measured (1) by high performance liquid chromatography (HPLC) at 30° C., and (2) by proton nuclear magnetic resonance (¹H NMR) spectroscopy at 300 megahertz using a 0.1 wt % solution of the CBD or CBDA extract in deuterated chloroform solution relative to a tetramethylsilane internal standard. The CBD or CBDA is in crystalline form and a 0.1 wt % solution of the extract in deuterated chloroform exhibits no detectable peak at 4.07 ppm, relative to a tetramethylsilane internal standard, as measured by ¹H NMR spectroscopy. In a more specific embodiment, the extract is a CBD extract and the extract exhibits a melting point as measured by differential scanning calorimetry (DSC) of 69-70° C.

The Cannabis plant material employed in the present methods is preferably one producing a high CBD and/or CBDA content extract. In one embodiment, the Cannabis plant material may be self-pollinating, i.e., monoecious, while in another embodiment, the Cannabis plant material be dioecious. In a specific embodiment, the Cannabis plant material contains less than 0.3% THC by dry weight and is within the definition of “industrial hemp” in Section 7606 of the Federal Agricultural Act of 2014.

In specific embodiments, the methods result in purified extracts having little or no detectable tetrahydrocannabinol (THC) or its acid form, tetrahydrocannabinolic acid (THCA). In more specific embodiments, the compositions or products of the invention contain greater than 50 wt %, greater than 60 wt %, greater than 70 wt %, greater than 80 wt %, greater than 90 wt %, greater than 95 wt %, greater than 97 wt %, greater than 99 wt %, or greater than 99.5 wt % CBD and/or CBDA, based on the weight of all cannabinoids in the compositions or products. In further embodiments, the compositions or products contains less than 3 wt %, less than 2 wt %, less than 1 wt %, less than 0.5 wt %, less than 0.1 wt %, less than 0.01 wt %, less than 0.004 wt %, or less than 0.001 wt %, THC and THCA, based on the weight of all cannabinoids in the compositions or products, when analyzed using high performance liquid chromatography (HPLC). In a further embodiment, the extract contains no detectable THC or THCA when analyzed using HPLC.

According to the invention, the purified, high content CBD/CBDA extract is isolated from Cannabis plant material by solvent extraction of CBDA, plus any CBD that may be present in the cannabis strain, preferably with methanol, ethanol, isopropanol, ethyl acetate, hexanes, heptanes, chloroform, or other lipophilic solvent. Alternatively, the high content CBD/CBDA extract is isolated from Cannabis plant material by extraction with pressurized liquid carbon dioxide, for example at or near ambient temperature, with supercritical or near supercritical carbon dioxide, or with pressurized superheated water at 100° C. to about 250° C., or using combinations of such pressurized technique in combination with one or more of the aforementioned solvent extractions at ambient pressure.

Following extraction, the extract is further treated by removing most or all of the solvent, for example, by heating under a protective blanket of nitrogen gas to a temperature at the boiling point of the solvent. The extract may be further heated at temperatures of about 110° C. to about 150° C. to decarboxylate the CBDA and form CBD, for example, for about 10 minutes to about 4 hours. In one embodiment, a CBDA extract is uniformly heated in a mineral oil bath, preferably at 110° C.-130° C. for 20-40 minutes, and provides CBD yields of greater than 75%. The heating is, in one embodiment, conducted in the dark. Care must be exercised to decarboxylate the CBDA to form CBD without decomposing the CBD product. The decarboxylation reaction can be conducted under vacuum and can be monitored by HPLC. The CBD product is in the form of a highly pure oil extract.

In an alternate embodiment, the Cannabis plant material, for example in finely ground form, may be first heated to decarboxylate CBDA therein to CBD, prior to extraction, using similar heating temperatures and times.

In one embodiment, the CBD/CBDA is extracted using ethanol, in which the CBD/CBDA is dissolved, followed by the addition of increasing amounts of water to the ethanol solution to crystallize the CBD/CBDA. Advantageously, a crystallized product, rather than an oil, is obtained.

Solvent extraction methods can produce an extract which includes an impurity exhibiting a detectable peak at 4.07 ppm, relative to a tetramethylsilane internal standard, as measured by ¹H NMR spectroscopy. This impurity can be isolated in the form of a white residue in methods according to the invention. In a specific embodiment, hydrocarbon (e.g., n-heptane) extracts of Cannabis sativa industrial hemp containing less than 0.3% of THC which are desolvated to form white crystallites of impure CBD and/or CBDA. The white crystals are then redissolved in ethanol, pressurized liquid carbon dioxide, for example at or near ambient temperature, or supercritical or near supercritical carbon dioxide, and a white impurity with a melting point of about 81° C. precipitates to form a separate solid scum which can be removed from the ethanol-CBD solution by sedimentation and decantation, filtration, centrifugation, or other recognized methods of separating solids from liquid solutions. These impurities are believed to be one or more long chain wax esters. Wax esters are commonly encountered in the cuticle of plant leaves and serve to protect the plant from dehydration. It has been discovered that the use of ethanol as an extraction liquid or pressurized liquid carbon dioxide, for example at or near ambient temperature, or supercritical or near supercritical carbon dioxide allows removal of such impurities from a CBD or CBDA extract as they are not soluble in ethanol or the indicated forms of carbon dioxide. In specific embodiments, once the impurity is removed, CBD and/or CBDA with less than 1 wt % organic impurities, less than 0.5% wt % organic impurities, or less than 0.1 wt % organic impurities, can be isolated by solvent evaporation. In a more specific embodiment, precipitated CBD and/or CBDA are obtained by progressively adding pure water, whereupon CBD and/or CBDA of unprecedented purity in crystalline form can be obtained after drying. The CBD crystals exhibit a melting point estimated by differential scanning calorimetry to be 69-70° C.

In another specific embodiment, the CBD/CBDA extract product may be sterilized by dissolving it in liquid carbon dioxide. The CBD/CBDA product is acidified by prolonged pressurization, followed by rapid depressurization with pressure swings, for example as described in U.S. Pat. No. 6,149,864. In a specific embodiment, the material is treated with supercritical fluid carbon dioxide at pressures in the range of from about 2000 to 3000 psi and temperatures preferably from about 2 to 45° C. for periods of from about 20 minutes to six hours, more preferably from about 0.5 to 2 hours. Agitation, pressure cycling, and the presence of water may enhance the sterilization method, which promotes diffusion of the supercritical fluid carbon dioxide to thereby alter the pH within the cells of any bacteria to kill the bacteria and/or rupture cells to kill the bacteria. The magnitude and frequency of the pressure cycling, as well as the process time and temperature, may vary according to the form of the CBD/CBDA material to be sterilized and the type of organisms to be killed.

In specific embodiments, the purified CBD and/or CBDA consists of CBD and/or CBDA, with no detectable impurities. In further embodiments, the purified CBD and/or CBDA consists essentially of CBD and/or CBDA, containing at least 99 wt % CBD and/or CBDA and less than 1 wt % of any organic impurity, or more specifically, containing at least 99.5 wt % CBD and/or CBDA and less than 0.5 wt % of any organic impurity, or even more specifically, at least 99.9 wt % CBD and/or CBDA and less than 0.1 wt % of any organic impurity.

In further embodiments, the purified CBD and/or CBDA is in the form of white, odorless crystals. Various Cannabis industrial hemp materials contain a number of odiferous components, including one, several or more of Δ-limonene, (−) limonene, linalool, (−) caryophyllene, α-humulene, caryophyllene oxide, terpinolene, Δ3-carene, (+) β-pinene, (−) camphor, α-pinene, (−) β-pinene, α-terpine, g-terpinene, geraniol, β-caryophyllene, (−) borneol, 1,4-cineole, 1,8-cineole (eucalyptol). The purified products which are odorless contain no detectable amounts of these components when the products are subjected to high performance liquid chromatography as described herein.

The purified CBD and/or CBDA extract, in oil or precipitated product form, can be administered transdermally with woven fiber patches, placed or dripped under the tongue where it is absorbed or otherwise taken into blood capillaries or used as a food additive or supplement, alone or with a diluent.

To avoid first pass removal by the liver and for faster action, the CBD can be vaporized, or formulated as a dry powder as discussed below, and inhaled.

The purified CBD and/or CBDA produced according to the invention can be used as an analytical standard in various therapeutic applications. Purity may be confirmed by analysis using High Performance Liquid Chromatography, Nuclear Magnetic Resonance Spectroscopy and/or Mass Spectrometry.

The purified CBD and/or CBDA may also be included as a component of a pharmaceutically acceptable composition for administration to a patient for a therapeutic effect in treatment of a disorder. In one embodiment, the pharmaceutically acceptable compositions of the invention may include CBD and/or CBDA in an amount above the placebo effect (including homeopathic compositions), up to and including 99 wt % pure CBD and/or CBDA. In specific embodiments, the compositions comprise about 1 to 90 wt %, 1 to 80 wt %, 10 to 70 wt %, 15-60 wt %, 20-60 wt %, or 25-50 wt % CBD and/or CBDA. The compositions may be in any conventional administration form, including solid unit dosage forms such as tablets, wafers, pellets, lozenges, solutions (for example, in water or ethanol), salves, creams, lotions, and the like, and may contain conventional additives, including pharmaceutical carriers, excipients, and the like.

The extracts, compositions and products of the invention may be administered to a mammal (human, rat, mouse, monkey, dog, cat, horse, etc.) for any one of various therapeutic effects for which CBD and/or CBDA are known in the art. In this regard, the extracts, compositions and products of the invention may be administered to provide anti-oxidant, anti-seizure, neuroprotective, anti-inflammatory, analgesic, anti-tumor, anti-stress, anti-psychotic, and/or anti-anxiety properties, among others. Treatment of multiple sclerosis, Parkinson's disease, alcohol abuse, tumor metastasis, stress, including, post-traumatic stress disorders, migraines, pain, concussion, anxiety, diabetes, and the like may be treated with the extracts, compositions and products of the invention.

In another embodiment, the CBD and/or CBDA extract can be formulated as a dry powder. In a specific embodiment, the CBD and/or CBDA extract can be formulated as a dry powder by forming a composition comprising a solution or emulsion of the extract, for example in water or a solvent, and a supercritical or near critical fluid, for example, carbon dioxide, and rapidly reducing the pressure on the composition, whereby droplets are formed, and passing the droplets through a flow of heated gas. Such methods are disclosed in the Sievers et al U.S. Pat. No. 6,630,121, incorporated herein by reference and known as the CAN-BD process. Further, the extract can be formulated with one or more additives, including, but not limited to sugars, polymers, amino acids, preservatives, and/or other excipients, and/or other active ingredients, for example, an antibiotic or vaccine, before forming the dry powder of the composition with the supercritical or near critical fluid. Suitable excipients include, but are not limited to, those used to increase solubility and/or dissolution rate of lipophilic cannabinoids, for example in lung fluid. Examples of suitable additives include, but are not limited to, myo-inositol, mannitol, sucrose, trehalose, leucine, lactose, tricine, sodium phosphate buffer, arginine, histidine, alanine, gelatin, lactalbumin hydrolysate, hydroxyethylstarch, maltodextrin, Tween 80, sodium citrate, phosphatidylcholine, alpha lipoic acid, methionine, glucosamine sulfate, phenylalanine polyethylene glycol (PEG), poly(lactic-co-glycolic acid) (PLGA), and polyvinylpyrrolidone (PVP), and the like. In a specific embodiment, one or more surfactants may be included in order to increase the solubility of the CBD and/or CBDA extract in the solution or emulsion. One suitable surfactant comprises lecithin, but one skilled in the art will appreciate that other conventional surfactants may also be employed. In specific embodiments, the weight ratio of CBD and/or CBDA to the excipients may be in a range of from about 1:100 to 100:1, more specifically, from about 1:50 to about 50:1, more specifically, from about 1:25 to about 25:1. In further embodiments, the weight ratio of CBD and/or CBDA to the excipients may be in a range of from about 1:25 to 1:1.

In specific embodiments, the composition for use in forming a dry powder comprises CBD and/or CBDA and mannitol, or mannitol and lecithin. The composition may also include a physiologically acceptable antioxidant, for example, methionine, or other additive, as desired.

In another specific embodiment, the composition for use in forming a dry powder comprises CBD and/or CBDA and polyvinylpyrrolidone (PVP). Various PVPs are commercially available at different molecular weights (weight average) and are suitable for use in the present compositions. In a specific embodiment, the PVP has a molecular weight in a range of from about 5000 to 500,000 Da, or, more specifically, from about 5000 to about 50,000 Da. In more specific embodiments, the PVP has a molecular weight of about 10,000 or 40,000 Da. The weight ratio of CBD and/or CBDA to PVP may be in a range of from about 1:100 to 100:1, more specifically, from about 1:50 to about 50:1, more specifically, from about 1:25 to about 25:1. In further embodiments, the weight ratio of CBD and/or CBDA to PVP may be in a range of from about 1:10 to 10:1. The CBD and/or CBDA and PVP may be dissolved in a solvent for the powder-forming process, for example, the CAN-BD process. Surprisingly, the dry powder compositions formed from a composition comprising CBD and/or CBDA, and PVP presents the CBD and/or CBDA in an amorphous form, and the powder contains no crystalline CBD and/or CBDA as measured by X-ray powder diffraction. The amorphous form may provide improved dissolution of the CBD and/or CBDA when administered in vivo for improved or expedited bioavailability or otherwise altered pharmacokinetic properties.

Formulation of the cannabinoid active pharmaceutical ingredients into dry powders facilitates the incorporation of such excipients which have been shown to increase solubility and dissolution rate, and therefore bioavailability, of lipophilic molecules such as cannabinoids. In one embodiment, the dry powder may be provided to include a particle fraction having an aerodynamic diameter effective to reach the deep lung for maximal absorption upon inhalation, for example, less than 5 μm, more specifically, in a range of 3-5 μm, as measured using an Andersen Cascade Impactor. In a more specific embodiment, at least 90 wt % of the particles have an aerodynamic diameter less than 5 μm, as measured using an Andersen Cascade Impactor.

The dry powder formulations are advantageous in exhibiting good storage stability and are much less susceptible to loss of material to packaging walls, in contrast to oils in which the active ingredients are dissolved in solution. In one specific embodiment, dry powder formulations are provided in single-dose blister packaging, for example formed of an aluminum-polymer laminate, to protect the powders from ambient moisture, bacterial and fungal ingress, and degradation by light.

The dry powders are suitable for administration as dry powder aerosols, for example, deliverable from dry powder inhalers like the Puff-Haler®, available from Sievers Biotech, or other such devices. Additionally, the dry powders may be compressed into a solid unit dosage form, for example, a tablet, wafer or pellet form, alone or, optionally, in compositions including one or more excipients or additives. In specific embodiments, the dosage form is a tablet having a thickness of at least about 1 mm or, more specifically, of at least 2 mm. In additional embodiments, the dosage form is a wafer having a thickness less than about 1 mm, or, more specifically, less than about 0.5 mm. The wafers, in one embodiment, are quick dissolving, i.e., they dissolve in less than about 2 minutes, less than about 1 minute, less than about 45 seconds, or less than about 30 seconds, when contacted with a liquid or saliva, and, in one embodiment, may be adapted for sublingual use when placed under the tongue of a patient.

In another embodiment, the invention is directed to adducts which comprise CBD or CBDA bonded to paramagnetic trivalent lanthanide (III) metal chelates (metal complexes). As noted above, CBD has been disclosed as useful in the treatment of a wide variety of disorders and conditions, including, inter alia, anxiety, post-traumatic stress disorder, cancer and epileptic seizures. As with any pharmaceutical compound, discovery of the optimal dosage form with which the compound can be delivered most effectively is highly desirable. Derivatization and methods of delivery that increase bioavailability, provide a time-release for consistent delivery throughout the day, or decrease the occurrence of side-effects or toxicity serve to enhance the inherent pharmacological properties of the compound and should be thoroughly explored and developed. The adducts of the invention provide a mechanism by which the properties of CBD and/or CBDA may be favorably altered through the formation of non-covalent bonds with another molecule, i.e., as a Lewis acid-base adduct. Non-covalent interactions have the potential to alter the effect that CBD has on the body through differences in solubility, absorption, and time-release, while not permanently or irreversibly altering the composition or properties of the CBD moiety within the adduct molecule. An ideal method for examining the formation of adducts is nuclear magnetic resonance (NMR) spectroscopy.

The inventive adducts which comprise CBD and/or CBDA bonded to paramagnetic trivalent lanthanide (III) metal chelates are useful for medical and diagnostic applications in concert with fluorescence spectroscopy. Paramagnetic transition metal complexes that are also coordinatively unsaturated can also form useful adducts with Lewis bases.

The lanthanide trivalent ions include cerium (Ce), praseodymium (Pr), neodymium (Nd), samarium (Sm), europium (Eu), gadolinium (Gd), terbium (Tb), dysposium (Dy), holmium (Ho), erbium (Er), thulium (Tm), and ytterbium (Yb). In a specific embodiment, the metal ion is europium (Eu) or ytterbium (Yb). Suitable ligands include, but are not limited to tris (1,1,1,2,2,3,3-heptafluoro-7,7-dimethyloctane-4,6-dionato); tris (1,1,1,2,2,3,3,7,7,7-decafluoro-4,6-heptanedionato); tris(1,1,1,2,2,3,3,3-heptafluoro-7,7-dimethyloctane-4,6-dionato); tris(1,1,1,5,5,5-hexafluoro-2,4-pentanedionato); perdeuterated tris(1,1,1,2,2,3,3-heptafluoro-7,7-dimethyloctane-4,6-dionato); perdeuterated tris(1,1,1,2,2,3,3-heptafluoro-7,7-dimethyloctane-4,6-dionato); tris (trifluoroacetyl-d-camphorato); tris(dipivaloylmethanato), and the like as disclosed in U.S. Pat. No. 3,846,333, incorporated herein by reference. An exemplary transition metal complexes are those formed with copper, such as bis((1,1,1,5,5,5-hexafluoro-2,4-pentanedionato)copper(II).

NMR spectroscopy is based upon the differential absorption of electromagnetic radiation by nuclear spin states of nuclei of atoms whose energy levels have been made non-degenerate by the presence of a strong magnetic field. A hydrogen nucleus contains one proton, which possesses an angular momentum and may exist in one of two possible spin states: +1/2 or −1/2. The energy levels of the two spin states are degenerate, and the spin produces a local magnetic field near the nucleus. Upon introduction of a strong external magnetic field by the instrument, the energy levels of the spin states diverge; the spin state aligned with the magnetic field becomes lower energy than the unaligned state and an energy gap between the spin states is produced. The instrument then irradiates the sample with electromagnetic radiation in the radio frequency range while holding it in a strong magnetic field created by a superconducting magnet. A number of the hydrogen protons that possess spin states of the lower energy level will absorb the radiation, reversing their spins to match those of the higher energy level. The frequency of radiation absorbed is recorded by the instrument and displayed as the difference in parts per million (ppm) from the absorption of an arbitrary standard, tetramethylsilane.

The local environment around the nucleus affects the size of the energy gap produced between spin state energy levels. Electronegative atoms such as oxygen draw electron density away from carbon and hydrogen, thus removing interference from the spins of the electrons, which produce their own local magnetic fields that obscure the nuclear magnetic field. Such hydrogens are said to be “deshielded” and are shifted downfield (to the left) in the NMR spectrum. The energy gap increases as deshielding increases, as the external magnetic field of the instrument is able to exert a more pronounced effect on the exposed nuclei. The ¹H NMR spectrum of CBD in the absence of adduct-forming coordinatively unsaturated paramagnetic lanthanide metal ions like europium(III) spans the region of 0.8 to 6.4 ppm, occupying upfield areas in which peaks corresponding to aliphatic, shielded hydrogen nuclei appear as well as downfield regions due to the electronegative hydroxyl groups.

CBD is capable of acting as a Lewis base and is capable of forming acid-base adducts with sufficiently strong Lewis acids, including the metal complexes formed between the metal ions and ligands described above. Specific Lewis acids include, but are not limited to, tris(1,1,1,2,2,3,3-heptafluoro-7,7-dimethyl-4,6-octanedionato)europium(III), (Eu(fod)₃), and tris(dipivaloylmethanato)ytterbium(III), (Yb(DPM)₃). Depending on the ratio of concentrations of CBD or other Lewis bases to the metal ion, and steric crowding and electronegativity considerations, adducts with more than one CBD or other Lewis base may form with molar ratios other than 1:1, such as 2:1. The ligands may be rapidly exchanging in steady state equilibria with other adducts when dissolved in solvents. Nevertheless, significant effects can be observed and taken advantage of in both the organic and inorganic parts of the adducts by measuring both the NMR proton (and/or the carbon-13 nuclei), and the fluorescence spectra in which excitation and emission spectra characteristics of complexed metal ions, i.e., europium (III) ions, aid in diagnosis and selective detection of cannabinoids like CBD in the presence of terpenoids such as myrcene, which has no oxygens and does not ordinarily bind to the trivalent metal ions such as Eu(III) or significantly affect its fluorescence.

The following Examples illustrate various aspects of the methods, compositions and products of the invention.

### EXAMPLE 1

### Ethanol Extraction of Dioecious Industrial Hemp

Extraction Procedure

A cultivar of OTTO-2 industrial hemp registered and developed by Centennial Seed Distributors, LLC, Lafayette, Colo., as part of the Colorado Department of Agriculture Research Program authorized in Section 7606 of the Federal Agriculture Act of 2014, was employed in this Example. Seeds were removed and leaves and blossoms were dried for cannabinoid analysis, with focus on cannabidiolic acid (CBDA) content in leaves and buds. Plant material (˜500 mg) was ground using a mortar and pestle, in triplicate. The pulverized sample was placed in a glass vial to which 10 ml of anhydrous ethanol was added. The vial was vortexed for 10 seconds, sonicated at setting “80” (maximum) for 15 minutes, and vortexed again for 10 seconds. A portion of the supernatant was removed and filtered through a cellulose acetate 0.2 micron syringe filter into a clean glass vial. The extract was diluted 2:13:10 (extract:methanol:water) into an HPLC vial.

HPLC Analysis

The HPLC method was taken from Backer et al (Innovative development and validation of an HPLC/DAD method for the qualitative and quantitative determination of major cannabinoids in cannabis plant material, J. Chromatogr. B, 877:4115-4124 (2009)), and was composed of the following gradient, with 50 mM ammonium formate, pH 5.11, as the aqueous component:

| Time (min.) | % Methanol | % Aqueous |
|---|---|---|
| 0 | 68 | 32 |
| 25 | 91 | 9 |
| 26 | 95 | 5 |
| 29 | 95 | 5 |
| 30 | 68 | 32 |
| 36 | 68 | 32 |

The parameters were as follows:

| Flow Rate | 0.3 | ml/min |
|---|---|---|
| Injection Volume | 30 | μl |
| Temperature | Room Temp. | |
| Detection Wavelength | 210 | nm |

| | |
|---|---|
| Column | C18 |

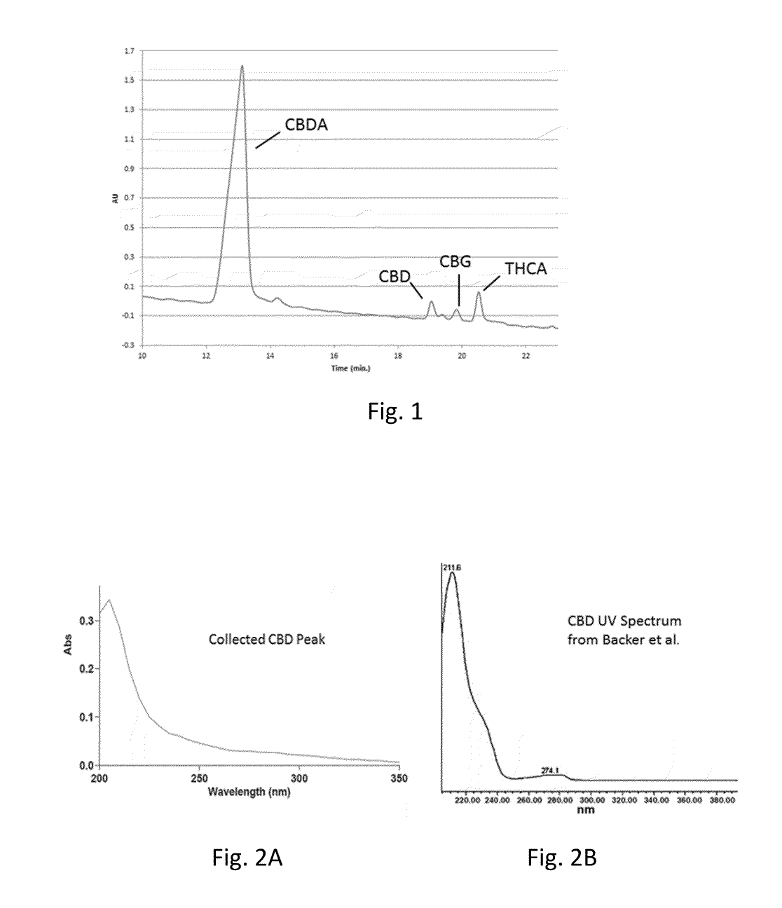

The chromatogram is shown in FIG. 1.

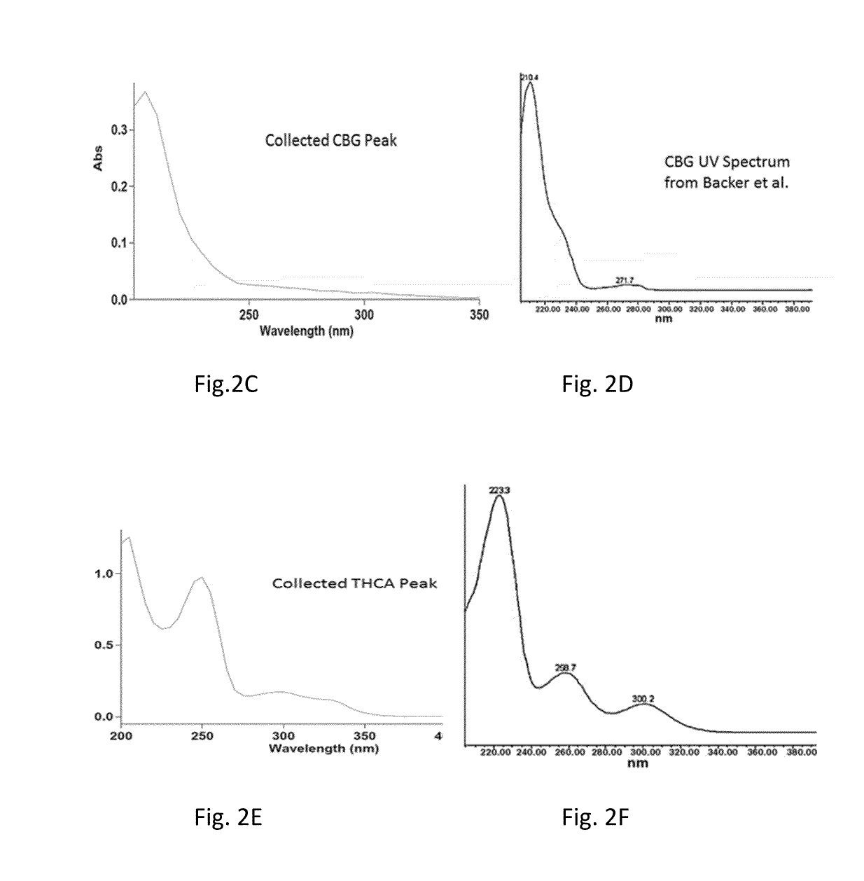

Because the three peaks with retention times of 19-21 minutes eluted very close together, the identity of each was examined by UV spectrophotometry, and the UV Spectra of minor peaks are shown in FIGS. 2A-2F. The presence of three peaks in the UV spectrum of this peak suggests that it is predominately the THCA cannabinoid acid, while the shapes of the other two UV spectra curves suggest that they are neutral cannabinoids.

The order of elution from the column corresponds with the order of elution from Backer et al.: CBD, CBG, and THCA, respectively. The weight percent of cannabinoids, based on the dry sample mass, were as follows:

| Cannabinoid | % of Dry Sample Mass |
|---|---|
| CBDA | 5.32 ± 0.15 |
| CBD | 0.09 ± 0.01 |
| CBG | 0.04 ± 0.00 |
| THCA | 0.14 ± 0.01 |

A different lot of OTTO-2 industrial hemp was analyzed independently by HPLC using the described procedure and determined to contain 12.6% CBDA but less than 0.03% THCA and <0.004% THC (0.001% being the detectable lower limit).

### EXAMPLE 2

### Analysis of Monoecious Hemp, High CBDA Content

Extraction Procedure

A cultivar of INFINITY™ (trademark Colorado Can, LLC) industrial hemp (defined by U.S. Federal law as having less than 0.3% THC) was employed in this Example. Seeds were removed and leaves and blossoms were dried for cannabinoid analysis, with focus on cannabidiolic acid (CBDA) content in leaves and buds. Plant material (502.3 mg) was ground using a mortar and pestle. The powder was placed in a glass vial to which 10 ml of anhydrous methanol was added. The vial was vortexed for 10 seconds, sonicated at setting “80” (maximum) for 15 minutes, and vortexed again for 10 seconds. A portion of the supernatant was removed and filtered through a cellulose acetate 0.2 micron syringe filter into a clean glass vial.

HPLC Analysis

The extract was diluted 570:1550 (extract:solvent) in an HPLC vial:

| | | |
|---|---|---|
| Extract | 570 | μl |
| Methanol | 440 | μl |
| Water | 440 | μl |
| Prazepam (1 mg/ml) | 100 | μl |
| Total | 1.55 | ml |

The HPLC method was taken from Backer et al. as described in Example 1, except using Prazepam (70 mg/L) as internal standard.

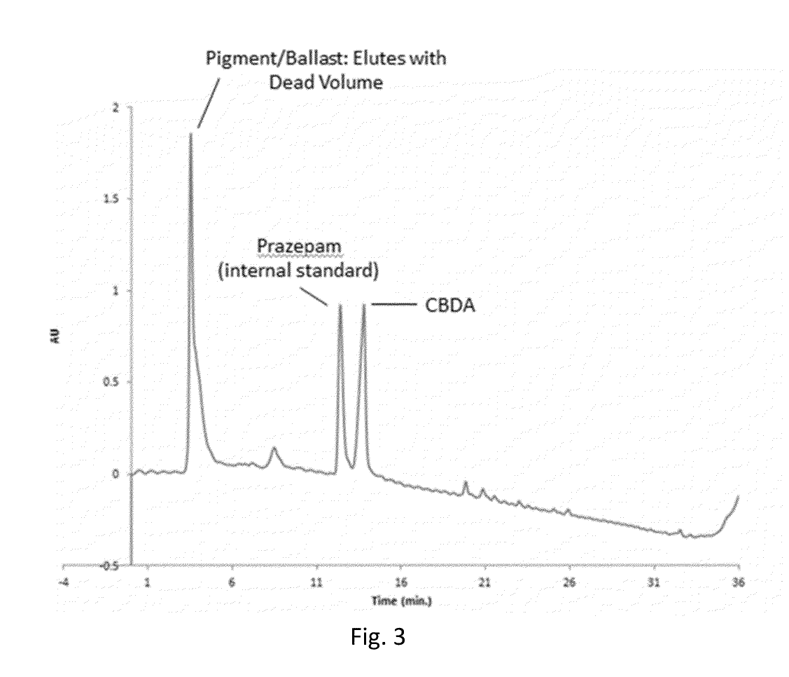

The chromatogram is shown in FIG. 3.

The peaks were integrated, and because a reference standard was not obtained for CBDA, its peak area was corrected using molar extinction coefficients at 210 nm found in the literature (Hazekamp et al, Chromatographic and spectroscopic data of cannabinoids from Cannabis sativa L, Journal of Liquid Chromatography and Related Technologies, 28:2361-2382 (2005)). The CBDA concentration was calculated using the formula:

$${C}_{CBDA} = \frac{{A}_{CBDA}}{{A}_{Praz}} \times \frac{{ɛ}_{Praz}}{{ɛ}_{CBDA}} \times {C}_{Praz}$$

where C_{CBDA} is the concentration of CBDA, A_{CBDA} is the integrated area under the CBDA peak, A_{Praz} is the integrated area under the prazepam peak, ε_{CBDA} is the molar extinction coefficient at 210 nm for CBDA, ε_{Praz} is the molar extinction coefficient at 210 nm for prazepam, and C_{Praz} is the spiked internal standard concentration of prazepam.

The mass percent of the identified cannabinoids in the sample are as follows:

| Cannabinoid | % by mass |
|---|---|
| CBDA | 0.61 |
| THCA | <0.001%* |
| THC | <0.001%* |

### EXAMPLE 3

### Pyrolysis of Methanol Extract of INFINITY™ Monoecious Hemp

Extraction Procedure

INFINITY™ plant material (502.3 mg) was ground using a mortar and pestle. The powder was placed in a glass vial to which 10 ml of anhydrous methanol was added. The vial was vortexed for 10 seconds, sonicated at setting “80” (maximum) for 15 minutes, and vortexed again for 10 seconds. A portion of the supernatant was removed and filtered through a cellulose acetate 0.2 micron syringe filter into a clean glass vial.

Pyrolysis

Aliquots (250 μl) of the filtered extract were placed into HPLC vials, and left open to air dry at room temperature in the dark for two days to an oily green solid. The vials were heated to 130° C. (except for a no-heat control sample) for 40 minutes in a heated desiccator oven, either at atmospheric pressure or under a vacuum of 635 torr, or in a heavy mineral oil bath under either a nitrogen atmosphere or open to the ambient air. The vials were removed, cooled, and their contents resuspended with methanol/water (50/50 v/v).

HPLC Analysis

The HPLC method was taken from Backer et al as described in Example 1.

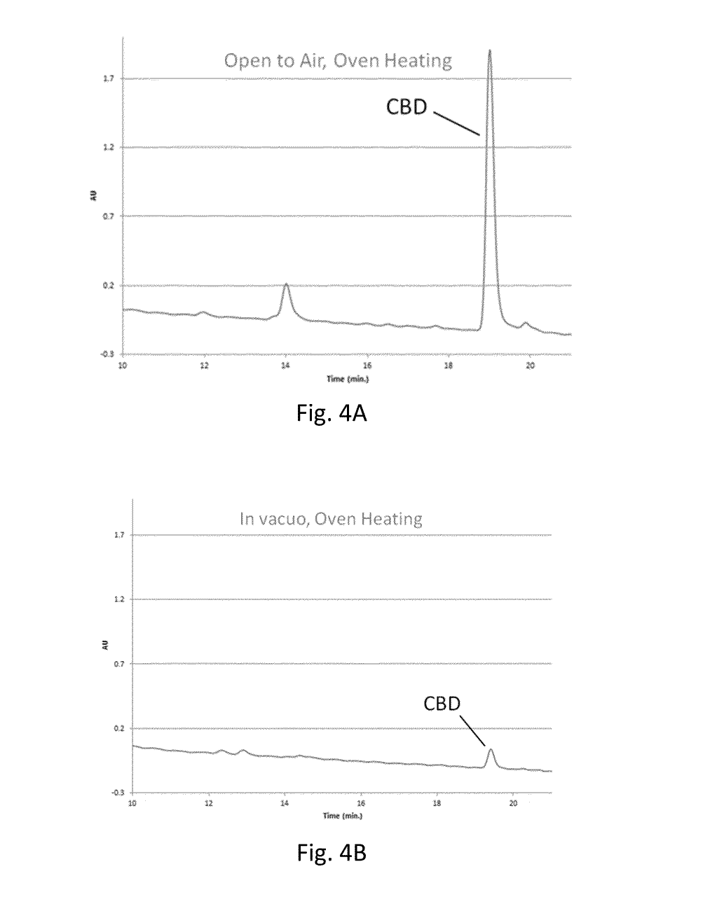

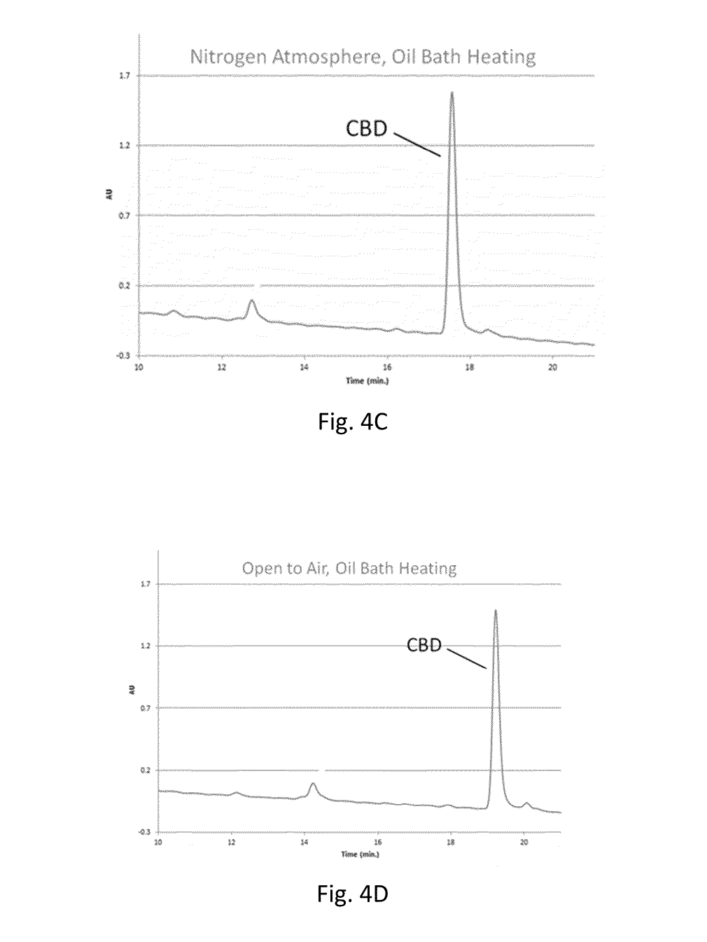

The chromatograms are shown in FIGS. 4A-4D.

| Heating Method | % yield CBD |
|---|---|
| Heated Desiccator (air) | 74.73 ± 2.75 |
| Heated Desiccator (635 torr vacuum) | 4.00 ± 0.42 |
| Mineral Oil Bath | 76.00 ± 2.19 |

None of the small peaks eluting during the liquid chromatography analysis had retention times corresponding with THC (above detectable amounts of >0.001%). Attempting to prevent the formation of the impurity by heating the samples under a slight (635 torr) vacuum was unsuccessful; the vacuum may have been sufficient to reduce the boiling point of the CBD (160° C. at 760 torr) and evaporate it from the sample vial under the 130° C. conditions. Heating the samples by immersion in a heavy mineral oil bath reduced the amount of the impurity by 37% as compared to heating in the heated desiccator oven; however, the yield of CBD was not significantly increased. Heating the samples by immersion in an oil bath under a nitrogen atmosphere did not further reduce the amount of the impurity. The concentration of oxygen in the nitrogen atmosphere could not be measured. A glovebox was purged with dry nitrogen for greater than 40 minutes until the relative humidity dropped from ambient (˜6%) to 1.6%.

### EXAMPLE 4

### Purification of CBD Using Near-Supercritical Carbon Dioxide

Sample Extraction

A partially purified polycrystalline CBD, imported under international treaty provisions (less than 100 mg for the first run, about 500 mg for the second run) was tied into a small bag made of white cotton muslin and placed into a stainless steel 10-ml Thar high-pressure vessel and attached to a valve, tee and 75 μm silica restrictor. The sample was extracted with CO₂ at 1380 psi into a clean glass vial. The first run was stopped after about 1 hour. During the second run, the run was interrupted partway through due to leaking of the Thar vessel; about 350 mg of sample remained in the bag. The extracted portion in the vial and the residue in the Thar vessel were analyzed by NMR.

Nuclear Magnetic Resonance (NMR) Analysis

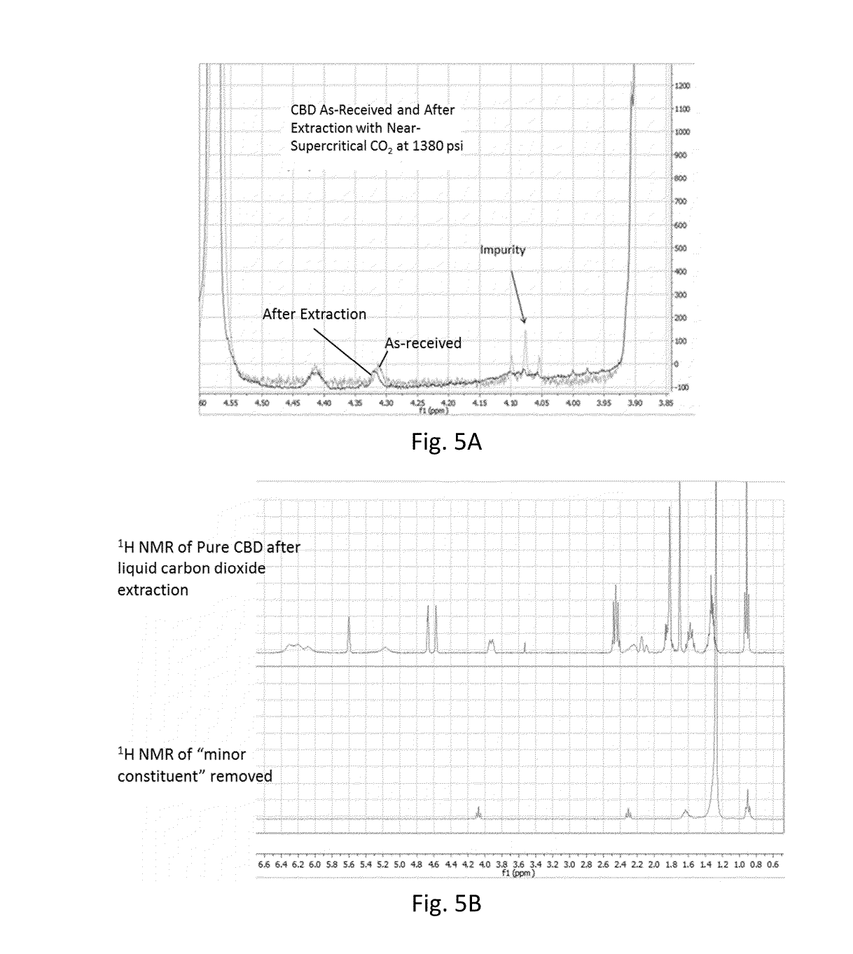

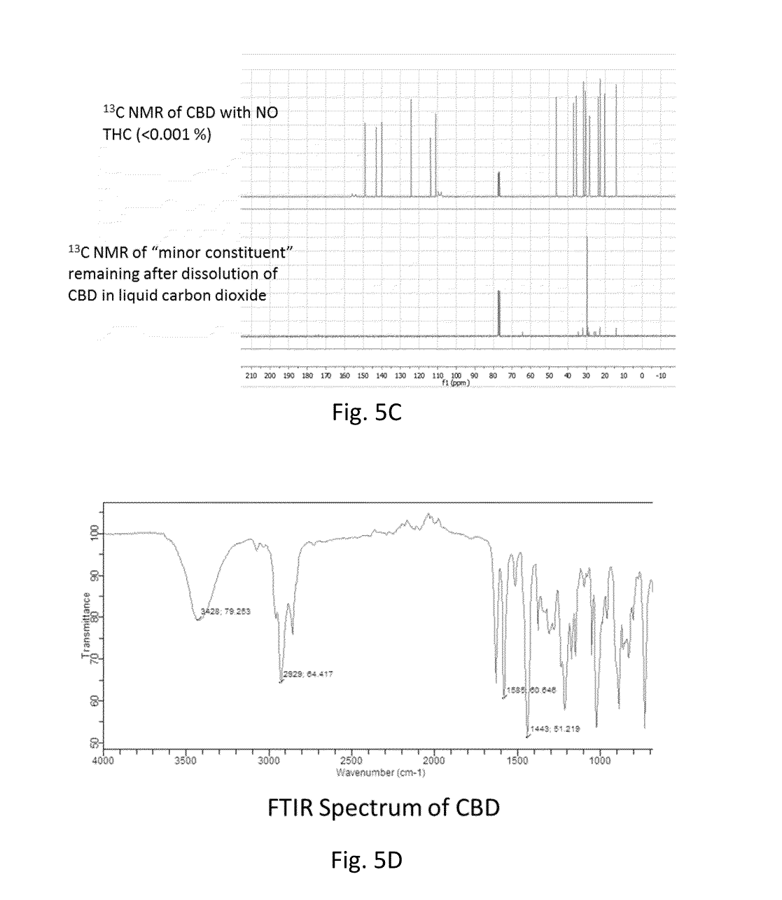

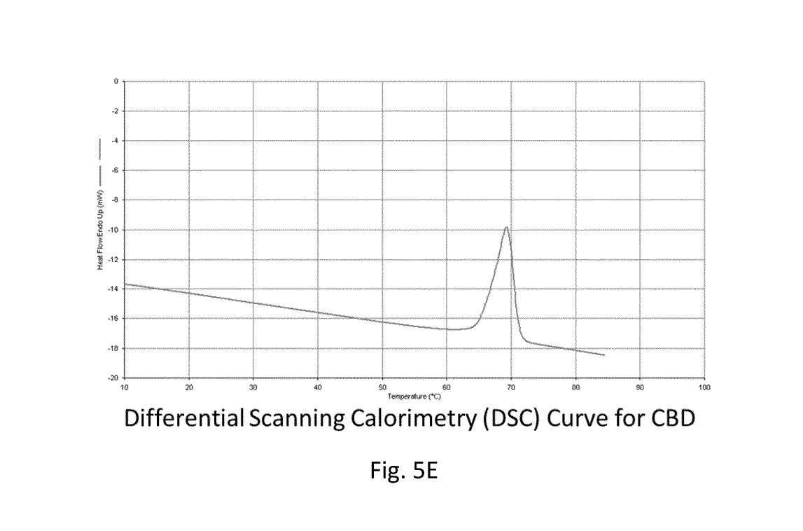

Samples were dissolved in deuterated chloroform (CDCl₃) (˜1 ml) and ¹H spectra was collected on a Bruker Avance-III 300 NMR spectrometer, at default settings, for 128 scans. The NMR Spectra are shown in FIG. 5A. Extracting the as-received sample with pressurized liquid CO₂ eliminates the impurity showing a triplet at 4.07 ppm relative to tetramethylsilane in an ¹H NMR spectrum from the extract. The CO₂ was allowed to slowly vaporize through a flow restrictor into a new vessel where pure CBD was collected and analyzed by ¹H and ¹³C NMR, Fourier-transform infrared spectroscopy (FTIR), differential scanning calorimetry (DSC), and chemical ionization mass spectrometry (CI-MS), shown in FIGS. 5B-5E. DSC was measured on a 3.0 mg sample encapsulated into aluminum pans designed for use with volatile substances and crimped to form a seal using a Perkin-Elmer Diamond Differential Scanning calorimeter and analyzed using Pyris software. The initial temperature was set at 0.00° C. for 5.0 min., and the temperature was then ramped from 0.00° C. to 350° C. at a rate of 20.00° C./min.

### EXAMPLE 5

### Purification of CBD Oil from Carbon Dioxide Extraction

CBD from a carbon dioxide extraction process on industrial hemp cannabis containing less than 0.3 wt % THC was collected in the form of a honey-like oil. The oil was first run through a silica column using toluene as the mobile phase, and then was dissolved in ethanol and winterized overnight in a freezer to allow removal of wax impurities. The solution was then run through a bed of activated carbon, a reverse phase (C18 modified) silica column using ethanol and acetic acid as the mobile phase, and, finally, another silica column. CBD was recrystallized as a white, odorless solid from an ethanol solution with water.

### EXAMPLE 6

### CBDA: Mannitol Inhalable Powder Using OTTO-2 Hemp Extract

Extraction Procedure

Pulverized OTTO-2 leaf and blossom dry plant material (1 g), previously measured to have 5.3% CBDA by weight, was ground using a mortar and pestle. The pulverized sample was placed in a glass vial to which 20-ml of anhydrous ethanol was added. The vial was vortexed for 10 seconds, sonicated at setting “80” (maximum) for 15 minutes, and vortexed again for 10 seconds. The ethanolic extract was removed and filtered through a cellulose acetate 0.2 micron syringe filter into a clean glass vial.

Sample Preparation

Mannitol (1 g) was dissolved in 15 ml of distilled and deionized H₂O. The filtered extract was added, mixed, and the resulting precipitate (chlorophyll, etc.) was gravity filtered using a Whatman filter, resulting in a yellow-green, slightly cloudy solution.

Carbon-Dioxide Assisted Nebulization with CAN-BD Processing

The solution was dried into a powder using a 50 ml Thar vessel with floating piston as the ethanolic solution sample chamber. A 50:50 ethanol:water mixture was placed into the sample pump to prevent precipitation should the piston seal leak.

The parameters were as follows:

| | | |
|---|---|---|
| Sample Flow Rate | 1 | ml/min |
| Sample Volume | ~30 | ml |
| CO₂ Pressure | 1350 | psi |
| N₂ Temperature | 45° | C. |
| N₂ Flow Rate | 20 | L/min |

| | |
|---|---|
| Filter | 0.45 μm cellulose acetate |

During the CAN-BD nebulizing and drying procedure, as described in U.S. Pat. No. 6,630,121, the carbon dioxide flow rate fluctuated in the range 1-3 ml/min, while the ethanol/water solution pressure was maintained at about 1440 psi. Some larger chunks of powder were observed collecting on the inner surface of the drying chamber. The pressure gauge connected to the drying chamber remained at about 1 psi during the procedure.

Powder Collection

The powder was pale yellow in color and flaked upon scraping. Several larger chunks were sitting loosely on top of the powder cake and were gently removed and excluded from the final powder sample. Powder mass=0.53 g, a 53% yield having an approximate 20:1 mannitol:CBDA weight ratio.

Wafer Formation

An aliquot of the powder (28.7 mg) was placed into an International Crystal Laboratories E-Z Press 7 mm diameter die for wafer formation, and a pressure of 800 psig was applied for one minute to form a wafer. Another aliquot of the powder (3.8 mg) was placed into a 4 mm diameter die, and a pressure of 1400 psig was applied for one minute to form a wafer.

### EXAMPLE 7

### CBD-PVP Amorphous Powder

Sample Preparation

PVP (0.25 g) of approximately 10,000 Da molecular weight and CBD isolate (0.25 g) were dissolved in 30 ml of methanol. Both solids dissolved completely into solution.

Carbon-Dioxide Assisted Nebulization with a Bubble Dryer (CAN-BD) Processing

The solution was dried into a powder with carbon-dioxide assisted nebulization with a bubble dryer (CAN-BD) using a 50 ml Thar vessel with floating piston as the methanolic solution sample chamber. Ethanol was placed into the sample pump to prevent precipitation should the piston seal leak.

The parameters were as follows:

| | | |
|---|---|---|
| Sample Flow Rate | 1 | ml/min |
| Sample Volume | 30 | ml |
| CO₂ Pressure | 1100 | psi |
| N₂ Temperature | 40° | C. |
| N₂ Flow Rate | 30 | L/min |

| | |
|---|---|
| Filter | 0.45 μm cellulose acetate |

During the CAN-BD nebulizing and drying process, the carbon dioxide flow rate fluctuated in a range of 3-4 ml/min, while the methanol solution pressure was ˜1120 psi. The pressure gauge connected to the drying chamber remained at ˜1 psi during the run. No plugging of the tee capillary was observed. The powder product was pure white in color and flaked upon scraping. The flakes collapsed into a free-flowing powder after being placed in a sample vial. Yield was 52%.

NMR Analysis

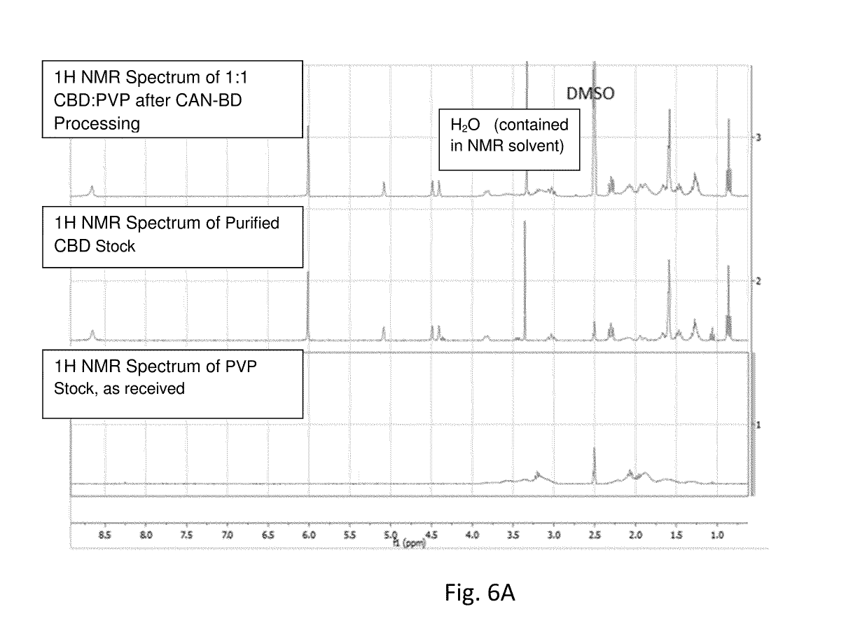

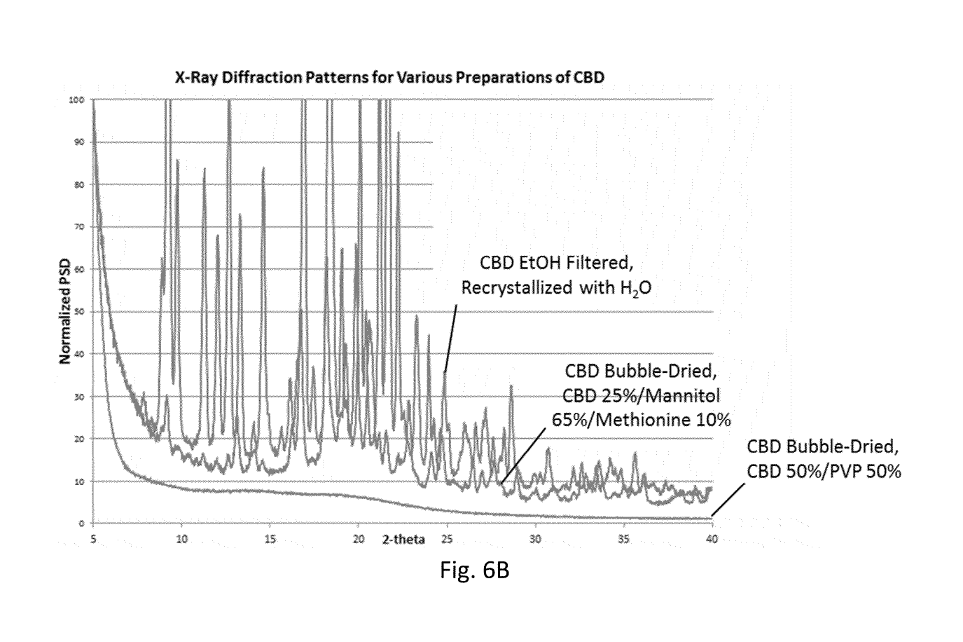

Samples of the powder were dissolved in dimethyl sulfoxide-d₆ and ¹H spectra were collected on a Bruker Avance-III 300 MHz NMR spectrometer, at default settings, for 32 scans. A comparison of the NMR analysis of the powder with that of the CBD prior to powder formation, FIG. 6A, indicates that the CBD remains chemically unchanged after processing.

X-Ray Diffraction (XRD) Analysis

Powder samples were lightly dusted onto a Vaseline-coated silicon zero-diffraction XRD plate (p-type, B-doped) of 24.6 mm diameter and 1.0 mm thickness. The samples were then analyzed on a Bruker D2 Phaser Diffractometer at 60 rpm. The XRD analysis indicates that the CBD/PVP bubble-dried powder is amorphous, in contrast to a previously prepared bubble-dried CBD/Mannitol/Methionine powder, FIG. 6B, and the purified, as-received CBD, which are both crystalline.

### EXAMPLE 8

### Adducts of Metal Complex Compounds with CBD

Adducts of metal complexes with CBD were formed with tris(1,1,1,2,2,3,3-heptafluoro-7,7-dimethyl-4,6-octanedionato)europium(III), (Eu(fod)₃), and with tris(dipivaloylmethanato) ytterbium(III), (Yb(dpm)₃). The following structures show (Eu(fod)₃) alone (a) and complexed with CBD (b):

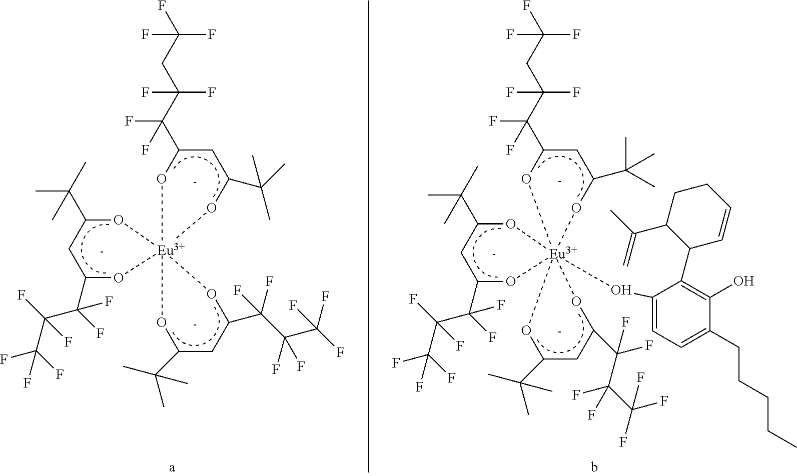

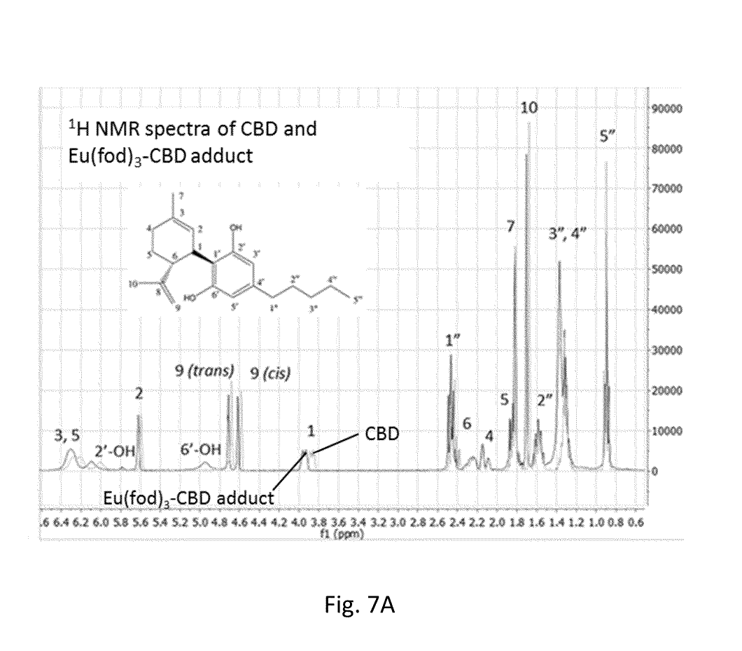

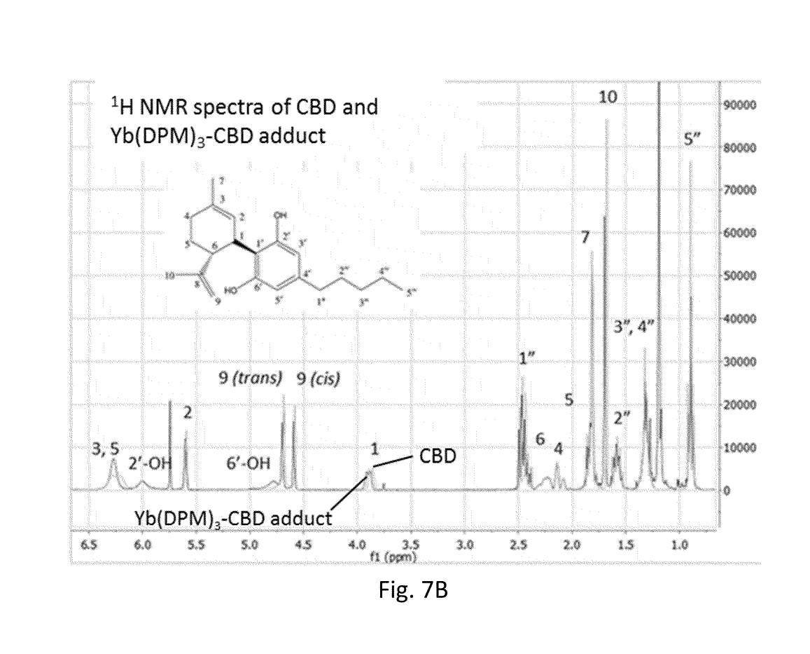

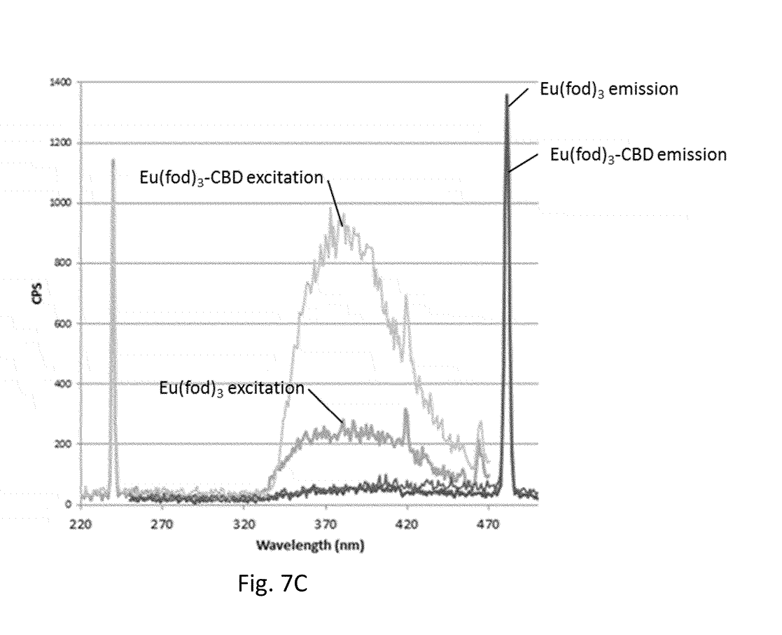

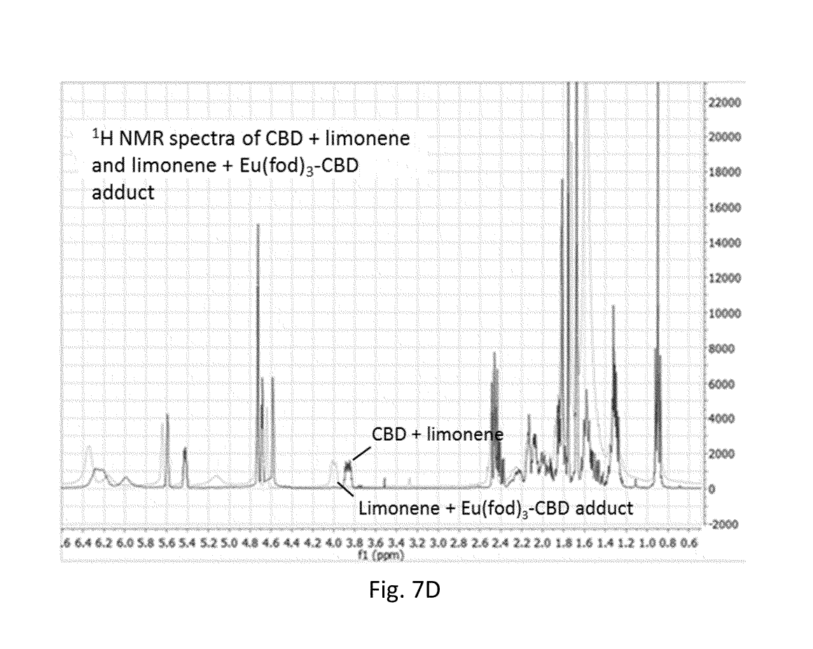

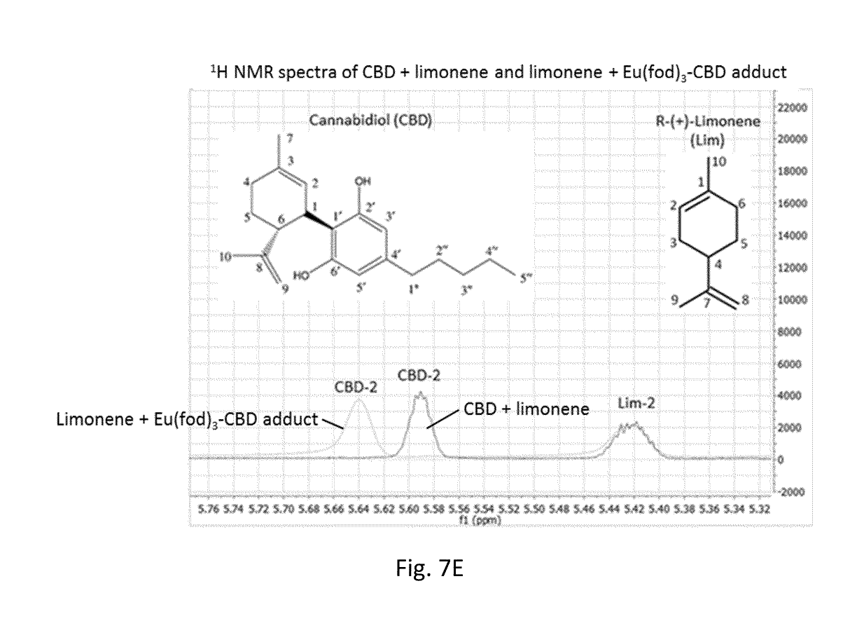

These formed adducts of metal complexes are new compositions of matter of the formula Eu(fod)₃-CBD and Yb(DPM)₃-CBD in which one of the hydroxyl groups in the CBD on the phenyl ring is reversibly bound to the coordinatively unsaturated europium(III) or ytterbium(III) ion. The adduct complexes were subjected to NMR analysis as shown in FIGS. 7A and 7B.

FIGS. 7A and 7B demonstrate that in the adducts, several of the CBD moiety peaks appear at different shifted downfield positions in the presence of these adduct-forming paramagnetic reagents, especially in the case of Eu(fod)₃-CBD, relative to free CBD that is not adducted by bonding to a paramagnetic metal ion. The aliphatic chain containing carbons 1″ through 5″ are remote enough from the paramagnetic Eu (III) ion to be relatively unaffected and the NMR peaks for these protons remain at the same positions as the CBD-only sample. Protons in the vicinity of the 2′-OH or 6′-OH display the greatest difference in shift positions, indicating that the acidic metal in added shift reagent has bound to the oxygen in the basic hydroxyl groups in the CBD molecule, and not to the hydrophobic carbon chain which has no oxygen atoms with unshared electron pairs.

When the non-adducted metal complex Eu(fod)₃ is irradiated and excited at 240 nanometers, a fluorescence emission band is emitted at 480 nanometers with relatively little background noise at other wavelengths. By itself, CBD does not significantly fluoresce when irradiated with ultraviolet light at 240 nanometers. But more intense violet light appears in other spectral bands when CBD is bound as a stable adduct to Eu(fod)₃, as shown in FIG. 7C. The phenomenon of new peaks appearing in the proton NMR in the presence of these Lewis acids occurs due to the paramagnetic nature of the trivalent europium chelate adduct. While a casual observation may make it seem that NMR peaks are “shifting”, actually a new compound and composition of matter has been formed with truly different NMR and fluorescence spectra. Fluorescence measurements and other independent single-crystal X-ray diffraction structural analyses of lanthanide beta-diketonate adducts have provided confirmation that new useful stable rare earth compounds, rather than physical admixtures, have been formed. Isolated regions of unpaired electrons within Eu(fod)₃ and Yb(DPM)₃ adducts produce an overall magnetic moment across the molecule that acts as a supplement to the external magnetic field provided by the NMR instrument. Protons in the vicinity of the bound shift reagent therefore experience a slightly greater magnetic field and the energy gap between spin states is increased, resulting in the absorption of higher frequency radiation and a downfield shifting of peaks. In contrast to covalently bound molecules in which the paramagnetic effect is accomplished via spin polarization through bonds, the Lewis acid-base adduct results in pseudocontact peakshifts, in which the effect is smaller and occurs due to spatial proximity to the paramagnetic center. This pseudocontact effect decays rapidly as distances from the paramagnetic center increase (1r³, where r is the distance between a particular proton and the bound shift reagent), which accounts for the observation of negligible differences in the magnetic environments for the peaks corresponding to hydrogens further removed from the hydroxyl groups.

The formation of a CBD-lanthanide(III) adduct is further confirmed by the selective shifting of CBD peaks even in solution with another compound. For example, in FIGS. 7D and 7E, NMR spectra of solutions of CBD and limonene, a common terpene found in Cannabis, are shown before and after the addition of Eu(fod)₃. The limonene peaks remain unbounded and therefore unshifted due to the absence of basic functional groups, while the protons near the hydroxyl groups in the CBD moiety within the adduct produce new peaks that appear downfield. Lanthanide(III) shift reagents will therefore not bind to every molecule present in a sample; only those compounds with Lewis basic moieties have the potential for adduct formation.

The various Examples and embodiments described herein are exemplary only and are not to be construed as limiting the scope of the invention defined by the following claims. Throughout this specification, when a range of conditions or a group of substances is defined with respect to a particular characteristic (e.g., temperature, pressure, amounts, and the like) of the present invention, the present invention relates to and explicitly incorporates every specific member and combination of subranges or subgroups therein. Any specified range or group is to be understood as a shorthand way of referring to every member of a range or group individually as well as every possible subrange and subgroup encompassed therein; and similarly with respect to any subranges or subgroups therein.

What is claimed is:

1. A cannabidiol (CBD) extract isolated from industrial hemp, comprising less than 0.5 wt % organic impurities as measured (1) by high performance liquid chromatography (HPLC) at 30° C., and (2) by proton nuclear magnetic resonance CH NMR) spectroscopy at 300 megahertz using a 0.1 wt % solution of the CBD extract in deuterated chloroform solution relative to a tetramethylsilane internal standard, wherein the CBD is in crystalline form, wherein a 0.1 wt % solution of the extract in deuterated chloroform exhibits no detectable peak at 4.07 ppm, relative to a tetramethylsilane internal standard, as measured by ¹H NMR spectroscopy at 300 megahertz, and wherein and the extract exhibits a melting point as measured by differential scanning calorimetry (DSC) of 69-70° C.

2. The extract of claim 1, comprising less than 0.1 wt % organic impurities as measured by the HPLC and ¹H NMR.

3. The extract of claim 1, comprising less than 0.004% of tetrahydrocannabinol (THC) and tetrahydrocannabinolic acid (THCA), based on total cannabinoids content, as measured by HPLC at 30° C.

4. The extract of claim 1, isolated from monoecious industrial hemp.

5. The extract of claim 1, isolated from dioecious industrial hemp.

6. A dry powder composition, comprising the extract of claim 1.

7. The dry powder composition of claim 6, wherein at least 90 wt % of the powder comprises particles have an aerodynamic diameter less than 5 μm, as measured using an Andersen Cascade Impactor.

8. The dry powder composition of claim 6, comprising at least one additional component selected from the group consisting of myo-inositol, mannitol, sucrose, trehalose, leucine, lactose, tricine, sodium phosphate buffer, arginine, histidine, alanine, gelatin, lactalbumin hydrolysate, hydroxyethylstarch, maltodextrin, Tween 80, sodium citrate, phosphatidylcholine, alpha lipoic acid, methionine, glucosamine sulfate, phenylalanine polyethylene glycol (PEG), poly(lactic-co-glycolic acid) (PLGA), and polyvinylpyrrolidone (PVP).

9. A dry powder composition, comprising polyvinylpyrrolidone and a cannabidiol (CBD) extract isolated from industrial hemp, wherein the CBD is amorphous.

10. The dry powder composition of claim 9, wherein the extract comprises less than 0.5 wt % organic impurities as measured (1) by high performance liquid chromatography (HPLC) at 30° C., and (2) by proton nuclear magnetic resonance CH NMR) spectroscopy at 300 megahertz using a 0.1 wt % solution of the CBD extract in deuterated chloroform solution relative to a tetramethylsilane internal standard, wherein a 0.1 wt % solution of the extract in deuterated chloroform solution exhibits no detectable peak at 4.07 ppm, relative to a tetramethylsilane internal standard, as measured by ¹H NMR spectroscopy at 300 megahertz.

11. A single unit dosage for oral delivery, comprising the dry powder composition of claim 6 in a compressed form.

12. The single unit dosage form of claim 11, comprising a wafer having a thickness less than 1 mm.

13. The single unit dosage form of claim 11, comprising a tablet having a thickness of at least 1 mm.

14. A method of producing the dry powder composition of claim 6, comprising mixing at least one carrier, an extract containing CBD or CBDA and a supercritical or near supercritical fluid, and rapidly reducing the pressure on the mixture, whereby droplets are formed, and passing the droplets through a flow of heated gas.

15. The method of claim 14, wherein the supercritical or near supercritical is carbon dioxide.

16. The method of claim 14, wherein the extract comprises CBDA and the method is conducted at a temperature of not more than about 40° C.

17. A method of purifying a cannabidiol (CBD) extract or a cannabidiolic acid (CBDA) extract in oil form, comprising dissolving the oil extract in near-supercritical carbon dioxide and removing a precipitated impurity exhibiting a peak at 4.07 ppm relative to a tetramethylsilane internal standard, as measured by proton nuclear magnetic resonance CH NMR) spectroscopy at 300 megahertz.

18. The method of claim 17, wherein the near-supercritical carbon dioxide is at a pressure of about 1300 1500 psig and a temperature less than about of not more than about 40° C.

19. A method of sterilizing a cannabidiol (CBD) extract or a cannabidiolic acid (CBDA) extract, comprising dissolving the extract in liquid carbon dioxide, pressurizing the solution to a pressure in a range of from about 2000 to 3000 psi, and repeatedly increasing and decreasing the pressure of the solution in the range of from about 2000 to 3000 psi.

20. The method of claim 19, wherein solution temperature is from about 2° C. to 45° C. and the pressure is increased and decreased for a period of from about 20 minutes to about six hours.

21. An adduct comprising cannabidiol (CBD) bonded to a paramagnetic trivalent lanthanide (III) metal chelate.

22. The adduct of claim 20, comprising CBD bonded to tris(1,1,1,2,2,3,3-heptafluoro-7,7-dimethyl-4,6-octanedionato)europium(III), (Eu(fod)₃), or to tris(dipivaloylmethanato)ytterbium(III), (Yb(DPM)₃).
